(19) United States
(12) Patent Application Publication
(10) Pub. No.: US 2014/0086867 A1
(43) Pub. Date: Mar. 27, 2014

(54) Systems, Compositions and Methods for the Treatment of Alopecia

(71) Applicant: Robert G. Matheny, Norcross, GA (US)

(72) Inventor: Robert G. Matheny, Norcross, GA (US)

(21) Appl. No.: 14/031,274

(22) Filed: Sep. 19, 2013

Related U.S. Application Data
(60) Provisional application No. 61/704,640, filed on Sep. 24, 2012.

(51) Int. Cl. A61K 35/38; A61K 45/06

(52) U.S. Cl. CPC A61K 35/38; A61K 45/06
USPC 424/85.1; 424/551; 424/93.7; 424/94.63; 424/130.1; 424/94.1; 604/22; 604/20

(57) ABSTRACT

A method of inducing or promoting hair growth on the scalp of a subject, comprising the steps of (i) providing an ECM composition including at least one ECM material, (ii) administering inciting event means to a target location on the scalp of the subject to induce an inciting event at the target scalp location, and (iii) administering a therapeutically effective amount of said ECM composition to the target scalp location. In some embodiments, the ECM composition includes at least one additional biologically active agent.

### CROSS-REFERENCES TO RELATED APPLICATIONS

This application claims the benefit of U.S. application Ser. No. 61/704,640, filed on Sep. 24, 2012.

### FIELD OF THE INVENTION

The present invention relates to methods for abating hair loss. More particularly, the present invention relates to extracellular matrix (ECM) compositions, and methods and systems using same, to abate hair loss and induce or promote hair growth.

### BACKGROUND OF THE INVENTION

As is well known in the art, androgenetic alopecia, better known as male-pattern baldness, is a clinical condition in which all or most of the hairs on a person's head are completely or partially lost. Alopecia affects approximately one-half of the world's male population and more than one-quarter of the female population. Nothwithstanding the high incidence of this condition, there is relatively little effective recourse available to those afflicted.

The exact mechanism for the hair growth disorder is not still fully understood, but it is speculated that alopecia occurs from dysfunction or disorder in relationship or interaction between hair follicle development and the hair cycle.

The hair cycle is divided into the following three phases: the growing phase (anagen), the transitional phase (catagen) and the resting phase (telogen). The anagen phase of a new hair commences at the moment it begins to actively grow. At that time there is very active differentiation of hair matrix cells in anagen hair follicles.

The catagen phase is the intermediate phase of the hair growth cycle. During the catagen phase, hair matrix cells cease hair production and the hair becomes a specific shape, i.e. a club.

The telogen phase is a resting phase during which hair follicles completely stop their activity and shrink.

If abnormal conditions are caused or introduced during the hair growth cycle, new hair may not regenerate upon alopecia. This thus means that the hair stem cells that are involved in hair growth and production no longer function. Non-functioning of the hair stem cells in the hair growth process results in non-functioning of hair papillae beneath hair follicles.

Considerable research and studies have thus been focused on stem cells and the relationship thereof to hair growth. For example, it has been reported that adult mice have multipotent stem cells that are related to morphogenetic signals involved in formation of multiple hair follicles, see Cell, vol. 104, pp. 233-245 (Jan. 26, 2001).

It has also been reported that follicular epithelial stem cells are present in hair follicle stem cells and that these epithelial stern cells function to modulate the hair cycle, see JID Symposium Proceedings, 8:28-38 (2003). In addition, it was reported that the interrelationship between the epithelium and mesenchyme is a crucial factor in formation of hair follicles.

Notwithstanding the abundance of research activities and development of hair growth methods, unfortunately, there is yet no therapeutically effective method capable of fundamentally preventing or treating alopecia. Alternative methods, which are currently available by modern medical techniques, include, for example, surgical procedures, such as hair transplantation and scalp plastic surgery, and various pharmacological (or drug) therapies.

Hair transplantation is one of the permanent solutions to balding, in which hair roots are removed from a donor region that still has normal functions, e.g., along the back and sides of the head, and transplanted into the scalp region where hair roots are dead.

Surgical operations, such as scalp plastic surgery, include, for example, scalp reduction surgery, scalp flap surgery and tissue extension or tissue expansion.

Examples of drug therapies include use of Minoxidil (a hypotensive drug) and Propecia (a prostate gland shrinking medication). Propecia and Minoxidil are the only two medications that are FDA approved for hair re-growth.

A major drawback associated with the noted drug therapies is that discontinuation of drug administration results in substantially no effects on treatment for hair loss. Therefore, the above-mentioned treatments are merely temporary means of treating hair loss rather than permanent means.

### SUMMARY OF THE INVENTION

The present invention is directed to extracellular matrix (ECM) compositions and methods and systems employing same to abate hair loss and/or induce or promote hair growth and/or hair follicle differentiation and development.

In a preferred embodiment, the ECM compositions include at least one ECM material.

In a preferred embodiment of the invention, the ECM material comprises mammalian extracellular matrix tissue selected from the group comprising small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof.

In some embodiments of the invention, the ECM compositions include at least one supplemental biologically active agent or composition, i.e. an agent that induces or modulates a physiological or biological process, or cellular activity, e.g., induces hair follicle differentiation and development.

In some embodiments of the invention, the biologically active agent comprises a growth factor selected from the group comprising a platelet derived growth factor (PDGF), epidermal growth factor (EGF), transforming growth factor-α(TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

In some embodiments, the biologically active agent comprises a cell selected from the group comprising a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

In some embodiments, the biologically active agent is selected from the group comprising collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

In some embodiments of the invention, the biologically active agent comprises a pharmacological agent or composition (or drug), i.e. an agent or composition that is capable of producing a desired biological effect in vivo, e.g., stimulation or suppression of an immune response, etc.

In some embodiments, the pharmacological agent comprises an anti-inflammatory agent.

In some embodiments, the pharmacological agent comprises a statin, i.e. a HMG-CoA reductase inhibitor.

In some embodiments of the invention, the ECM material comprises a decellularized ECM material.

In a preferred embodiment, the ECM material is decellularized via the use of supercritical carbon dioxide and a rapid vessel depressurization step.

In some embodiments of the invention, there is provided an ECM delivery apparatus and system for delivering an ECM composition of the invention to a subject to modulate the hair growth cycle, i.e. abate hair loss and/or induce or promote hair growth and/or hair follical differentiation and development.

In some embodiments, the ECM delivery system is structured to deliver an ECM composition directly to a target site or location on a subject, for example, the crown region on the scalp of a subject.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration depicting the layers of human skin;

FIGS. 2A and 2B are illustrations of a human scalp, depicting regions thereof;

FIGS. 3A-3E are illustrations of perietal, occipital, frontal temporal and vertex ECM grafts that are configured to mimic selective portions of a subject's scalp, according to various embodiments presented herein;

FIG. 4 is a perspective view of one embodiment of an ECM composition delivery system, in accordance with the invention;

FIG. 5 is a perspective view of another embodiment of an ECM composition delivery system having a microneedle array, in accordance with the invention;

FIG. 6 is a perspective view of one embodiment of an ECM microneedle, in accordance with the invention; and

FIG. 7 is a perspective view of another embodiment of a microneedle, in accordance with the invention.

### DETAILED DESCRIPTION OF THE PREFERRED EMBODIMENT

Before describing the present invention in detail, it is to be understood that this invention is not limited to particularly exemplified compositions, apparatus, systems, structures or methods as such may, of course, vary. Thus, although a number of compositions, apparatus, systems and methods similar or equivalent to those described herein can be used in the practice of the present invention, the preferred compositions, apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an agent” includes two or more such agents and the like.

Further, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10”, as well as “greater than or equal to 10” is also disclosed.

### Definitions

The term “inciting event”, as used herein, means and includes an event or action that causes cellular damage or injury and/or induces cellular migration or a cascade of cytokines and/or growth factors and/or other molecules, and/or other cellular activities that are associated with tissue remodeling or wound repair.

The term “graft”, as used herein, means and includes a portion of a tissue or organ configured for placement on host tissue of a subject.

The terms “extracellular matrix”, “ECM” and “ECM material” are used interchangeably herein, and mean and include a collagen-rich substance that is found in between cells in mammalian tissue, and any material processed therefrom, e.g. decellularized ECM. According to the invention, the ECM material can be derived from a variety of mammalian tissue sources, including, without limitation, small intestine submucosa (SIS), urinary bladder submucosa (UBS), stomach submucosa (SS), central nervous system tissue, epithelium of mesodermal origin, i.e. mesothelial tissue, dermal extracellular matrix, subcutaneous extracellular matrix, gastrointestinal extracellular matrix, i.e. large and small intestines, tissue surrounding growing bone, placental extracellular matrix, ornamentum extracellular matrix, cardiac extracellular matrix, e.g., pericardium and/or myocardium, kidney extracellular matrix, pancreas extracellular matrix, lung extracellular matrix, and combinations thereof. The ECM material can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM material can also be derived from basement membrane of mammalian tissue/organs, including, without limitation, urinary basement membrane (UBM), liver basement membrane (LBM), and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM material can also be derived from other sources, including, without limitation, collagen from plant sources and synthesized extracellular matrices, i.e. cell cultures.

The term “acellular”, as used herein, means extracellular matrix compositions that are at least 80% decellularized, such that the extracellular matrix composition is 80% without cells and/or cellular remnants. In some exemplary aspects described herein, the term “acellular” can thus refer to extracellular matrix compositions that are at least 90% decellularized such that the extracellular matrix composition is at least 90% without cells and/or cellular remnants.

Thus, as used herein, the term “acellular” can refer to extracellular matrix compositions that are decellularized at levels of 80%, 81%, 82%, 83%, 84%, 85%, 86%, 87%, 88%, 89%, 90%, 91%, 92%, 93%, 94%, 95%, 96%, 97%, 98%, 99%, 100%, and any percentages falling between these values.

The terms “biologically active agent” and “biologically active composition” are used interchangeably herein, and mean and include agent that induces or modulates a physiological or biological process, or cellular activity, e.g. induces cellular migration or differentiation.

The terms “biologically active agent” and “biologically active composition” thus mean and include, without limitation, the following growth factors: platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platlet derived growth factor (PDGF), tumor necrosis factor-α (TNA-α), and placental growth factor (PLGF).

The terms “biologically active agent” and “biologically active composition” also mean and include, without limitation, human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, growth factors, cytokines, cell-surface associated proteins, cell adhesion molecules (CAM), angiogenic growth factors, endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPS), enzymes and enzyme inhibitors, anticoagulants and/or antithrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of tissue, and vasodilating agents.

The terms “pharmacological agent”. “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti VGEFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include, without limitation, the following antiobiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The term “emulsion”, as used herein, means a mixture in which a first ECM material is dispersed within a second ECM material, with the first ECM material being immiscible with the second ECM material. The “emulsions” described herein can refer to either oil-in-water type emulsions or water-in-oil type emulsions.

The term “suspension”, as used herein, means a mixture in which a solid ECM material, such as, for example and without limitation, particulate ECM, is dispersed (suspended) in a fluid ECM material, such as, for example and without limitation, ECM gel or ECM liquid.

The term “biocompatible”, as used herein, means a device or material that is substantially non-toxic in an in vivo environment, and is not substantially rejected by a recipient's physiological system, i.e. non-antige.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, fuaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “ECM composition”, as used herein, thus means and includes an ECM material in combination with a “biologically active agent” and/or a “pharmacological agent” and/or any additional agent or component identified herein.

The term “therapeutically effective”, as used herein, means that the amount of the ECM composition administered to a subject is of sufficient quantity to abate hair loss or induce or promote hair growth, or hair follicle differentiation or development, or ameliorate one or more causes, symptoms, or sequelae of a disease or disorder associtaed with hair loss or abated hair growth. Such amelioration only requires a reduction or alteration, not necessarily elimination, of the cause, symptom, or sequelae of the disease or disorder.

The terms “delivering,” “delivery,” and “deliver” are used interchangeably herein, and mean the application of an ECM composition of the invention to a site on a subject, wherein the ECM composition imparts its therapeautic properties. In some exemplary aspects described herein, the term “delivering” can thus refer to injection of the ECM composition subdennally, spraying of the ECM composition to a site, misting of the ECM composition to a site, applying by hand the ECM composition to a site, use of a device to apply the ECM composition to a site, and the use of microneedles to apply the ECM composition to a site.

The terms “prevent” and “preventing” are used interchangeably herein, and mean and include reducing the frequency or severity of a disease, pathological condition, or disorder associtated with hair loss or abated hair growth. The terms do not require an absolute preclusion of the disease or condition. Rather, the terms include decreasing the chance for disease occurrence.

The terms “treat” and “treatment” are used interchangeably herein, and mean and include medical management of a patient with the intent to abate hair loss or induce or promote hair growth, or hair follicle differentiation or development, or ameliorate one or more causes, symptoms, or cure, ameliorate, stabilize, or prevent a disease, pathological condition, or disorder associated with hair loss or abated hair growth. The terms include “active treatment”, i.e. treatment directed specifically toward the improvement of a disease, pathological condition, or disorder associated with hair loss or abated hair growth, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder associated with hair loss or abated hair growth, “preventative treatment”, i.e. treatment directed to minimizing or partially or completely inhibiting the development of the associated disease, pathological condition, or disorder associated with hair loss or abated hair growth, and “supportive treatment”, i.e. treatment employed to supplement another specific therapy directed toward the improvement of the associated disease, pathological condition, or disorder associated with hair loss or abated hair growth.

The terms “optional” and “optionally” mean that the subsequently described event, circumstance, or material may or may not occur or be present, and that the description includes instances where the event, circumstance, or material occurs or is present and instances where it does not occur or is not present.

The term “comprise” and variations of the term, such as “comprising” and “comprises,” means “including, but not limited to” and is not intended to exclude, for example, other additives, components, integers or steps.

The terms “subject”, “patient” and “recepient” are used interchangeably herein, and mean and include any warm blooded mammal.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

As discussed above, the present invention is directed to extracellular matrix (ECM) compositions, and methods and systems using same, to abate hair loss and induce hair growth. As will readily be appreciated by one having ordinary skill in the art, the present invention substantially reduces or eliminates the disadvantages and drawbacks associated with prior art methods and apparatus for abating hair loss and/or inducing hair growth.

### ECM Compositions

In a preferred embodiment, the ECM compositions include at least one ECM material.

In some embodiments of the invention, the ECM compositions comprise sterilized acellular ECM compositions that are preferably formed by contemporaneously sterilizing and decellularizing an isolated ECM material.

Suitable sterilized acellular ECM compositions and methods for making same are set forth in Co-Pending Application Nos. 13/480,140, 12/707,427, 13/480,205, and 1/747,028; which are incorporated by reference herein in their entirety.

In addition to decellularizing the ECM material, as described herein, the rapid depressurization of the ECM material can also be employed to incorporate desired sterilants and selective biologically active agents into the ECM material.

In exemplary aspects, the sterilized acellular ECM composition can comprise any known ECM component or material, including, for example and without limitation, mucosal layers and components, submucosal layers and components, muscularis layers and components, dermis, and/or basement membrane layers and components.

It is contemplated that a disclosed sterilized acellular ECM composition can comprise an ECM material obtained from any mammalian tissue source, including, for example and without limitation, stomach tissue (e.g., stomach submucosa (SS)), small intestinal tissue (e.g., small intestinal submucosa (SIS)), large intestinal tissue, bladder tissue (e.g., urinary bladder submucosa (UBS)), liver tissue (e.g., liver basement membrane (LBM)), heart tissue (e.g., pericardium), lung tissue, kidney tissue, pancreatic tissue, prostate tissue, mesothelial tissue, fetal tissue, a placenta, a ureter, veins, arteries, tissue surrounding the roots of developing teeth, and tissue surrounding growing bone.

It is further contemplated that a disclosed sterilized acellular ECM composition can comprise an ECM material obtained from ECM components or materials of one or more mammals including, for example and without limitation, humans, cows, pigs, dogs, sheep, cats, horses, rodents, and the like. Thus, it is contemplated that a disclosed sterilized acellular ECM composition can comprise ECM components or materials from two or more of the same mammalian species, such as, for example and without limitation, two or more cows, two or more pigs, two or more dogs, or two or more sheep.

It is further contemplated that a disclosed sterilized acellular ECM composition can comprise ECM components or materials from two or more different mammalian species, such as, for example and without limitation, a pig and a cow, a pig and a dog, a pig and a sheep, or a cow and a sheep. It is still further contemplated that a disclosed sterilized, acellular ECM composition can comprise ECM components or materials obtained from a first tissue source, such as, for example and without limitation, SIS, from a first mammal, as well as ECM components or materials obtained from a second tissue source, such as, for example and without limitation, SS, from a second mammal.

In various aspects, a disclosed sterilized acellular ECM composition can be produced in any suitable shape, including, for example and without limitation, a substantially flat sheet, a deformable sturcture, a deformable or moldable structure that conforms to the shape of the scalp, or a structure whose borders follow the outline of the various scalp regions a cylindrical tube. The ECM composition can also be formed as a multi-laminate and/or substantially spherical structure.

It is contemplated that a disclosed sterilized acellular ECM composition can also be produced in any suitable form, including, for example and without limitation, a solid, liquid, gel, particulate, emulsion, or suspension form.

In one exemplary aspect, it is contemplated that a disclosed sterilized acellular ECM composition can comprise an outer layer of solid ECM material that encloses an inner layer of liquid, particulate, emulsion, suspension, and/or gel ECM material.

In another exemplary aspect, it is contemplated that a disclosed sterilized acellular ECM composition can comprise one or more types of particulate ECM materials that are suspended within an ECM gel to form an ECM suspension. In this aspect, it is contemplated that the particulates within a disclosed ECM suspension can have a diameter ranging from about 5 μm to about 300 μm, with an average diameter ranging from about 90 μm to about 100 μm.

It is further contemplated that the percentage of gel within a disclosed ECM suspension can range from about 5% to about 50%, while the percentage of particulate within a disclosed ECM suspension can range from about 50% to about 95%.

In exemplary aspects, the ECM gel of a disclosed ECM suspension can comprise a hydrolyzed ECM material. In these aspects, it is contemplated that the ECM gel of a disclosed ECM suspension can comprise ECM that is greater than about 50% hydrolyzed, more preferably, greater than about 70% hydrolyzed, and, most preferably, greater than about 90% hydrolyzed.

In one exemplary aspect, the ECM gel of a disclosed ECM suspension can comprise ECM that is approximately 100% hydrolyzed.

It is still further contemplated that the ECM components of the suspension can comprise at least one of the following: glycoproteins, such as, for example and without limitation, fibronectin and laminan; glycosaminoglycans, such as, for example and without limitation, heparan, hyaluronic acid, and chondroitin sulfate; and growth factors, thereby providing additional bioavailability for native cellular components.

It is contemplated that the ECM components of the suspension can provide a structural and biochemical microenvironment that promotes cell growth and stem cell attraction following administration of a disclosed ECM composition.

It is further contemplated that the ECM gel of a disclosed ECM suspension can function as a bulking agent that preserves a desired biomechanical environment until the cells of the subject can begin producing their own ECM.

It is still further contemplated that the desired biomechanical environment that is preserved by the ECM gel can substantially correspond to a biomechanical environment in native tissue.

Thus, it is contemplated that the ECM gel of a disclosed ECM suspension can have an elastic modulus that is substantially equal to the elastic modulus of a target site on a subject.

In exemplary aspects, the elastic modulus of the ECM gel of a disclosed ECM suspension can range from about 5 kPa to about 50 kPa, and, more preferably, from about 10 kPa to about 15 kPa.

It is contemplated that one or more of the aforementioned biologically active agents can be incorporated into the ECM material to impart selected properties to the resulting sterilized, acellular ECM composition.

In this aspect, it is contemplated that the biologically active agent(s) can be selected to replace or supplement components of the ECM material that are lost during processing of the ECM material. For example, and as described below, the biologically active agent(s) can comprise growth factors, cytokines, proteoglycans, glycosaminoglycans (GAGs), proteins, peptides, nucleic acids, small molecules, drugs, or cells.

It is further contemplated that the biologically active agent(s) can be selected to incorporate non-native components into the ECM material. For example, the biologically active agent(s) can comprise, for example and without limitation, growth factors for recruiting stem cells, angiogenic cytokines, and anti-inflammatory cytokines.

It is still further contemplated that the biologically active agent(s) can comprise one of the aforementioned pharmaceutical agents, such as statins, corticosteroids, non-steroidal anti-inflammatory drugs, anti-inflammatory compounds, anti-arrhythmic agents, and the like.

It is still further contemplated that the biologically active agent(s) can comprise nanoparticles, such as, for example and without limitation, silver nanoparticles, gold nanoparticles, platinum nanoparticles, iridium nanoparticles, rhodium nanoparticles, palladium nanoparticles, copper nanoparticles, zinc nanoparticles, and other metallic nanoparticles.

It is still further contemplated that the biologically active agent(s) can comprise metallic compounds. In one exemplary aspect, the biologically active agent(s) can be selected to pharmaceutically suppress the immune response of a subject following implantation of the resulting ECM composition into the body of a subject.

It is thus contemplated that the biologically active agent can comprise one or more growth factors, including, for example and without limitation, transforming growth factor-β −1, −2, or −3 (TGF-β −1, −2, or −3), fibroblast growth factor-2 (FGF-2), also known as basic fibroblast growth factor (bFGF), vascular endothelial growth factor (VEGF), placental growth factor (PGF), connective tissue growth factor (CTGF), hepatocyte growth factor (HGF), Insulin-like growth factor (IGF), macrophage colony stimulating factor (M-CSF), platelet derived growth factor (PDGF), epidermal growth factor (EGF), and transforming growth factor-α (TGF-α).

It is further contemplated that the biologically active agent can comprise one or more cytokines, including, for example and without limitation, stem cell factor (SCF), stromal cell-derived factor-1 (SDF-1), granulocyte macrophage colony-stimulating factor (GM-CSF), interferon gamma (IFN-gamma), Interleukin-3, Interleukin-4, Interleukin-10, Interleukin-13, Leukemia inhibitory factor (LIF), amphiregulin, thrombospondin 1, thrombospondin 2, thrombospondin 3, thrombospondin 4, thrombospondin 5, and angiotensin converting enzyme (ACE).

It is further contemplated that the biologically active agent can comprise one or more proteoglycans, including, for example and without limitation, heparan sulfate proteoglycans, betaglycan, syndecan, decorin, aggrecan, biglycan, fibromodulin, keratocan, lumican, epiphycan, perlecan, agrin, testican, syndecan, glypican, serglycin, selectin, lectican, versican, neurocan, and brevican.

It is further contemplated that the biologically active agent can comprise one or more glycosaminoglycans, including, for example and without limitation, heparan sulfate, hyaluronic acid, heparin, chondroitin sulfate B (dennatan sulfate), and chondroitin sulfate A.

It is further contemplated that the biologically active agent can comprise one or more proteins, peptides, or nucleic acids, including, for example and without limitation, collagens, elastin, vitronectin, versican, laminin, fibronectin, fibrillin-1, fibrillin-2, plasminogen, small leucine-rich proteins, cell-surface associated protein, cell adhesion molecules (CAMs), a matrikine, a matrix metalloproteinase (MMP), a cadherin, an immunoglobin, a multiplexin, cytoplasmic domain-44 (CD-44), amyloid precursor protein, tenascin, nidogen/entactin, fibulin I, fibulin II, integrins, transmembrane molecules, and osteopontin.

It is still further contemplated that the biologically active agent can comprise one or more statins, including, without limitation, cerevastatin, atorvastatin, fluvastatin, lovastatin, mevastatin, pitavastatin, pravastatin, rosuvastatin, and simvastatin.

It is further contemplated that the biologically active agent can comprise one or more anti-inflammatory agents, including, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, connethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredoneacetate, ibufenac, ibuprofen, ibuprofenaluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

It is further contemplated that the biologically active agent can comprise one or more steroids, including, without limitation, (e.g., andranes (e.g., testosterone), cholestanes (e.g., cholesterol), cholic acids (e.g., cholic acid), corticosteroids (e.g., dexamethasone), estraenes (e.g., estradiol), and pregnanes (e.g., progesterone).

It is further contemplated that the biologically active agent can comprise an exogenous protein, such as those normally found in mammalian ECM. According to the invention, the protein can comprise a collagen, a proteoglycan, a glycosaminoglycan (GAG) chain, a glycoprotein, a growth factor, a cytokine, a cell surface associated protein, a cell adhesion molecule (CAM), an angiogenic growth factor, an endothelial ligand, a matrikine, a matrix metalloprotease, a cadherin, an immunoglobulin, a fibril collagen, a non-fibrillar collagen, a basement membrane collagen, a multiplexin, a small-leucine rich proteoglycan, decorin, biglycan, a fibromodulin, keratocan, lumican, epiphycan, a heparan sulfate proteoglycan, perlecan, agrin, testican, syndecan, glypican, serglycin, selectin, a lectican, aggrecan, versican, nuerocan, brevican, cytoplasmic domain-44 (CD-44), macrophage stimulating factor, amyloid precursor protein, heparin, chondroitin sulfate B (dermatan sulfate), chondroitin sulfate A, heparan sulfate, hyaluronic acid, fibronectin (Fn), tenascin, elastin, fibrillin, laminin, nidogen/entactin, fibulin I, fibulin II, integrin, a transmembrane molecule, platelet derived growth factor (PDGF), epidermal growth factor (EGF), transforming growth factor-α (TGF-α), transforming growth factor-β (TGF-β), fibroblast growth factor-2 (FGF-2) (also called basic fibroblast growth factor (bFGF)), thrombospondin, osteopontin, angiotensin converting enzyme (ACE), or a vascular epithelial growth factor (VEGF). This list is not intended to be exhaustive.

In one optional aspect, in order to provide a sterilized, acellular ECM composition into a particulate form, the method can comprise cutting the ECM composition into pieces having desired lengths.

In another aspect, the method can optionally comprise freeze-drying the pieces of the ECM composition.

In an additional aspect, the method can optionally comprise grinding the frozen, hydrated pieces of the ECM composition and then passing the pieces of the ECM composition through a filter system, e.g., screen, until ECM particulate of a desired size is isolated.

In a further optional aspect, the method can comprise rehydrating the ECM particulate with sterile saline or other sterile, biocompatible fluid to form an ECM suspension, as described herein.

According to the invention, the ECM compositions can be delivered or administered to a subject via manual means, i.e. by hand or with an applicator, or via a pressurized container, i.e. mist or spray application, or via an ECM delivery system of the invention. In accordance with at least one embodiment of the invention, upon administration of an ECM composition to a target region on a subject's scalp afflicted with stagnent hair growth or hair loss, (i) cell function is restored, whereby hair loss is substantially reduced or eliminated and (ii) the hair growth cycle is re-initiated, whereby hair color is also restored.

In a preferred embodiment, the administration of an ECM composition of the invention to the scalp of a subject is preceded by or administrated simultaneously with an “inciting event”. As set forth above, the term “inciting event” means and includes an event or action that causes cellular damage or injury and/or induces cellular migration or a cascade of cytokines and/or growth factors and/or other molecules, and/or other cellular activities that are associated with tissue remodeling or wound repair.

According to the invention, various means and modalities can be employed to provide or induce an inciting event within a subject's scalp, including, without limitation, the application of ultrasound, radio frequency (RF), laser, ultraviolet and infrared energy. Various mechanical means, such as piercing the scalp and, hence, tissue can also be employed.

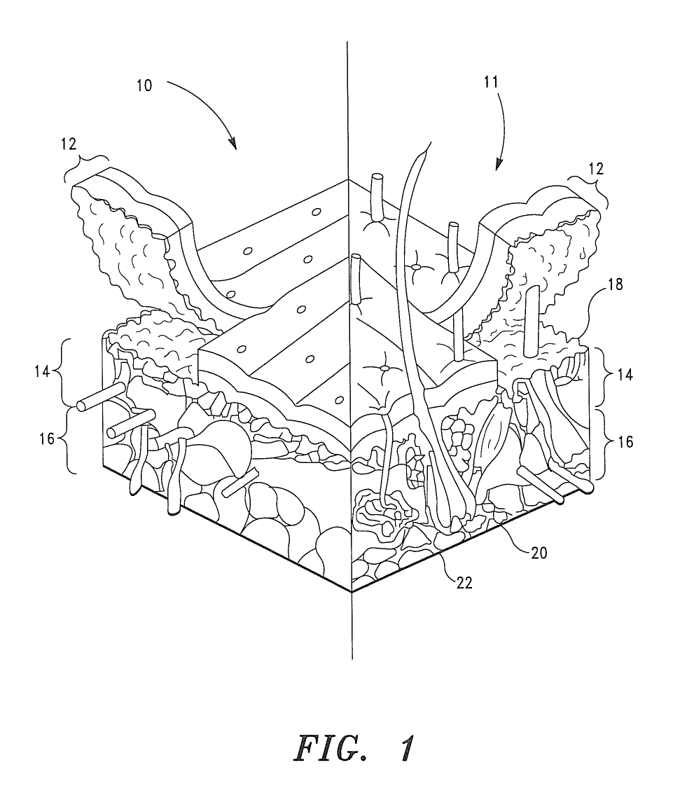

Referring now to FIG. 1, there is shown an illustratation of two sections of a scalp or skin, depicting the layers therein with respect to a hairless skin layer 10 and a thin skin layer 11. As illustrated in FIG. 1, the sub-layers within the skin layers 10, 11 comprise the epidermis 12, the dermis 14, and the subcutis/hypodermis 16. The thin skin layer 11 (with hair) includes the dermal papillae 18, hair follicle 20, and eccrine sweat gland 22.

According to the invention, the depth to which an ECM composition can be administered to the scalp of a subject comprises topical and intradermal administration to the epidermis 12, intradermal administration to the dermis 14, and subcutaneous administration to the subcutis/hypodermis 16. Topical administration to the epidermis 12 corresponds to ECM composition delivery to the scalp surface, whereas intradermal administration to the epidermis 12 corresponds to a depth of approximately 50 μm up to 1.5 mm. More preferably, when the ECM composition is administered intradermally to the epidermis 12, it is delivered to a depth of approximately 100 μm up to 1 mm.

If the ECM composition is to be administered to the dermis 14, the ECM composition is preferably administered topically or to a depth of approximately 2.0 mm (0.0 mm-2.0 mm). More preferably, the ECM composition is administered at depths from approximately 0.2 mm-1.5 mm. Most preferably, the ECM composition is administered at depths from approximately 0.5 mm-1.0 mm.

### ECM Delivery Systems

According to the invention, various delivery apparatus and systems can be employed to deliver the ECM compositions to the scalp of a subject, including traditional injection means, such as needles. Other delivery means include coated microneedles, i.e. microneedles having an ECM composition deposited thereon, as well as microneedles that include internal reservoirs that are configured to receive an ECM composition therein and disperse the ECM composition therefrom.

A medicated patch or bandage can also be employed to deliver an ECM composition topically and potentially concomitantly with an injection means. This particular embodiment can be useful in situations where it is desired to deliver the ECM composition to multiple layers of the skin.

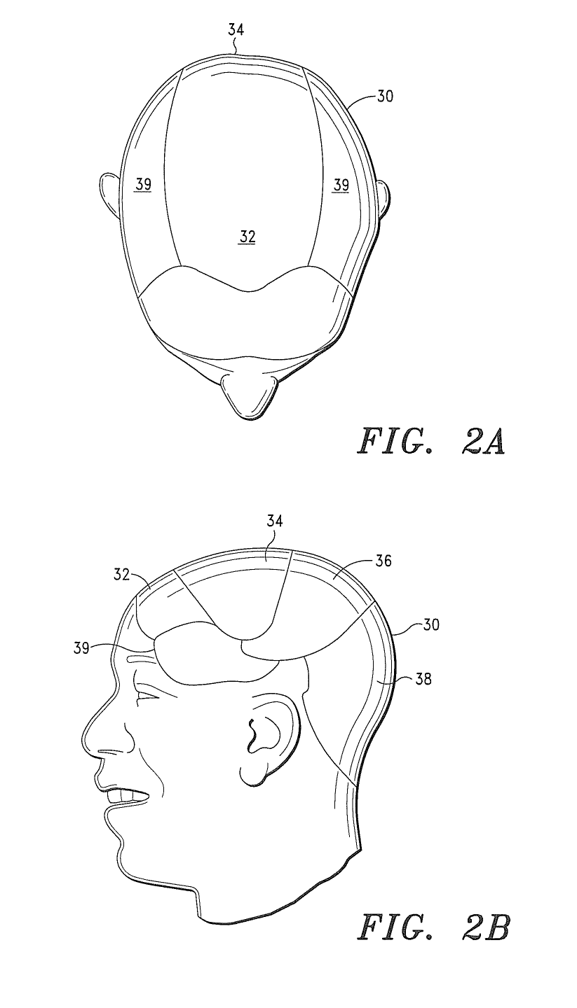

Referring now to FIGS. 2A and 2B, there are shown illustrations of a subject's head depicting regions of the subject's scalp 30. As illustrated in FIG. 1, the scalp regions 30 include the frontal region 32, the vertex region 34, the parietal region 36, the occipital region 38, and the temporal region 39. It should be noted that the temporal region 39, is located on both the left and right side of the scalp 30, equaling two distinct regions.

As stated, in one embodiment of the invention, the ECM composition delivery means comprises a graft comprising or including an ECM composition of the invention. According to the invention, the ECM graft can be composed of multilayered ECM and can contain one or more of the aforementioned additives.

In some embodiments of the invention, the ECM graft includes at least one, more preferably, a plurality of microneedles (described below) on a skin contact surface.

According to the invention, the ECM graft can comprise a deformable structure that conforms to the shape of the scalp 30 when positioned thereon or a structure that is cut into a shape that mimics the region of the scalp 30 that is balding.

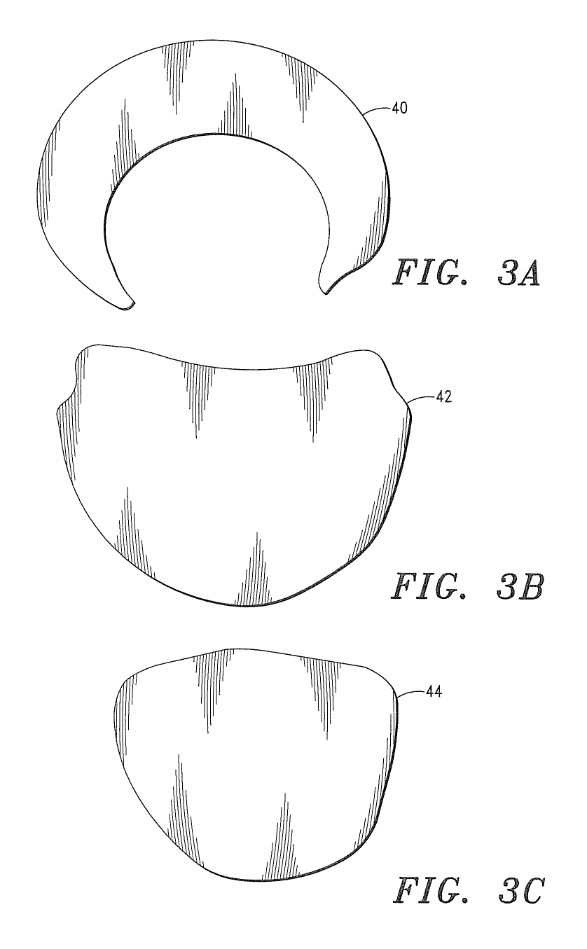

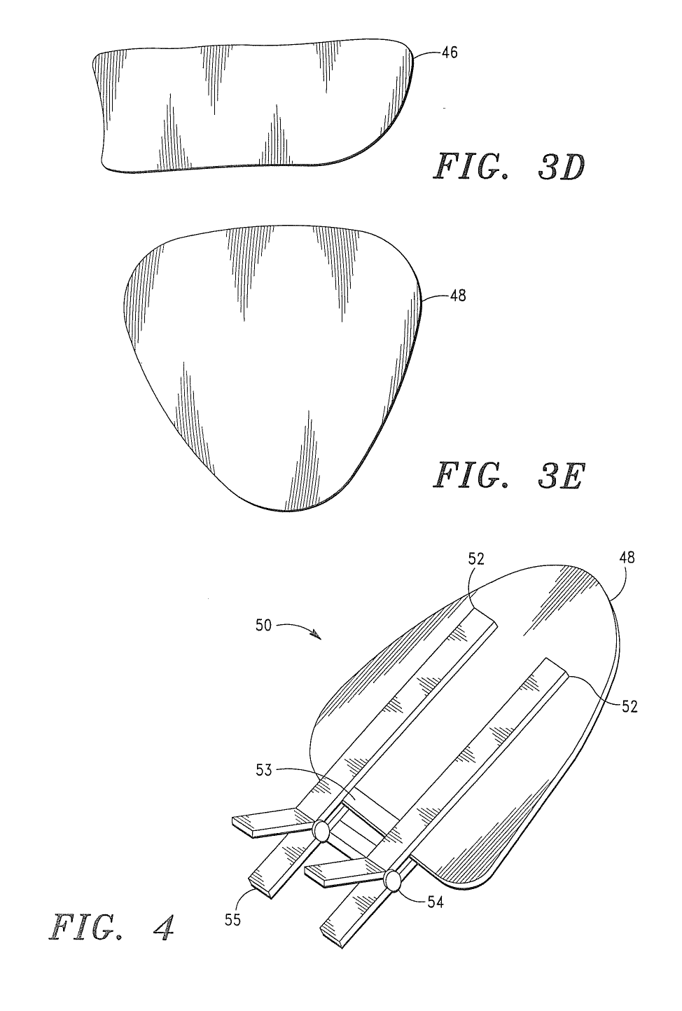

Referring now to FIGS. 3A-3E, there are shown embodiments of various ECM grafts of the invention. Referring first to FIG. 3A, there is shown one embodiment of a parietal region ECM graft 40, which preferably mimics the shape of the parietal region 36.

FIGS. 3B, 3C, 3D and 3E, illustrate embodiments of an occipital region ECM graft 42, a frontal region ECM graft 44, a temporal region ECM graft and a vertex region ECM graft 48.

Referring now to FIG. 4, there is shown one embodiment of an ECM composition delivery system 50. In some embodiments of the invention, the ECM composition delivery system 50 includes two upper prongs 52 that help to support the ECM composition graft, in this instance a vertex portion ECM graft 48, during and prior to delivery. According to the invention, the upper prongs 52 can be connected via a crossbar 53 that functions to stabilize the ECM composition delivery system 50. In other embodiments, the crossbar 53 can be repeated over the length of the upper prongs 52 at particular intervals to further strengthen the stabilization of the ECM composition delivery system 50.

The ECM composition delivery system 20 further comprises lower prongs 55 that are located below the ECM composition graft 48. According to the invention, the noted lower prongs 55 can be substantially the same width and length as the upper prongs 52, and in other embodiments they can be a different size relative to other constraints.

In one embodiment, the lower prongs 55 are connected at a pivot point 54 that connects the lower prongs 55 to the upper prongs 52. In some embodiments of the invention, the pivot point 54 can contain a spring or other mechanism that applies force to the prongs 52, 55 so that they grip the ECM composition graft 48.

In one embodiment, the upper prongs 52 are positioned and adapted to rise at an angle near the pivot point 54 such that there is an increased range of motion to allow for a greater opening range of the ECM composition delivery device. The noted angled upper prongs 52 allow for easier removal of the ECM composition delivery system post delivery.

According to the invention, the ECM composition delivery system 50 can comprise various conventional materials, including, but not limited to, stainless steel, nitnol, tantalum, titanium, chromium, magnesium, PLA, PEEK, and propylene. The ECM composition delivery system 50 can also comprise other materials that would be obvious to one of ordinary skill in the art.

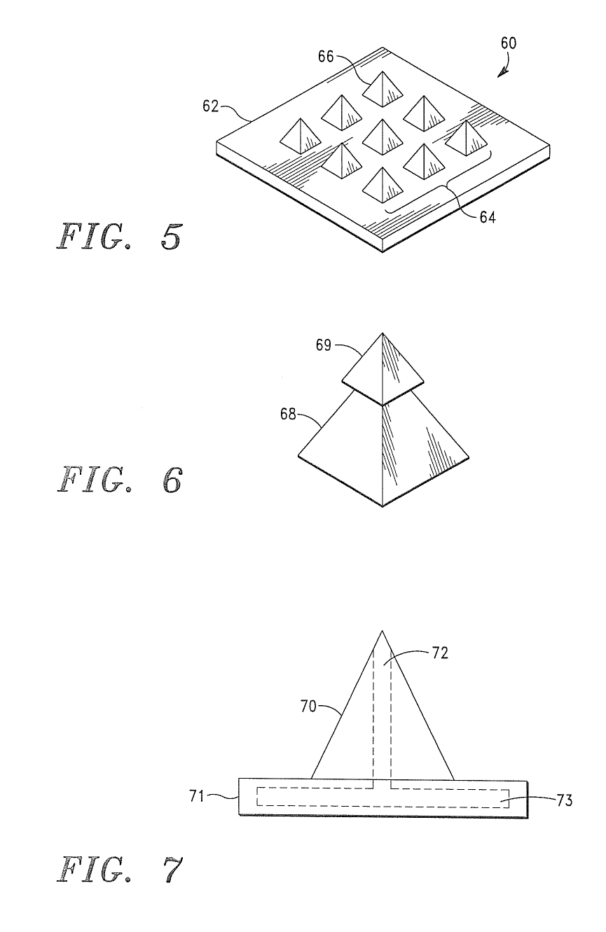

Referring now to FIG. 5, there is shown another embodiment of an ECM composition delivery system 60. As illustrated in FIG. 5, in this embodiment, the delivery system 60 comprises a microneedle delivery member 62.

Preferably, the microneedle delivery member 62 includes a microneedle array 64 that consists of one or more microneedles 66. According to the invention, the microneedle array 64 can comprise various sizes and shapes, including the size and shapes of the aforementioned ECM grafts.

In some embodiments of the invention, at least one, more preferably, all of the microneedles 66 (or microneedle array 64) are coated with an ECM composition of the invention.

The delivery of an ECM composition through the use of a coated microneedle array 64 facilitates delivery of an ECM composition through the skin of a subject, and also allows for accelerated recovery, increased control of location of delivery, reduced trauma to the surrounding tissue, and reduced cost associated with the procedure.

According to the invention, the microneedle array 64 and/or microneedles 66 can comprise various materials. Preferably, the microneedle array 64 and microneedles 66 comprise biocompatible materials. In some embodiments, the microneedle array 64 and/or microneedles 66 comprise a biodegradable material, such as magnesium, whereby in the event of breakage, any material that remains within the dermis slowly degrades and results in little trauma or inflammatory response from the patient.

In some embodiments, the entire microneedle array 64 is composed of an ECM composition. In some aspects, the ECM microneedles degrade and deliver the ECM composition to the desired region upon engagement to host tissue, e.g. scalp. In some aspects, the ECM microneedles are designed and configured to break off sub- or intra-dermal and deliver the ECM composition to the desired region. It is further contemplated that any single part or all parts of the microneedle array 64 can comprise the ECM composition.

Referring now to FIG. 6, there is shown another embodiment of a microneedle 68 that has an ECM composition presented as a tip 69 that can be implanted sub- or intra-dermal. According to the invention, the ECM composition tip 69 can comprise a solid, film, gel, suspension, sheet, etc.

Referring now to FIG. 7, there is shown another embodiment of a microneedle 70 that includes a base 71 and an internal lumen 72 that is configured to receive an ECM composition therein and disperse the ECM composition therefrom. According to the invention, the microneedle base chamber 73 can be constructed so that each microneedle 70 within a microneedle array has a separate microneedle chamber, or so that all the microneedles within the microneedle array share a single microneedle chamber, or that a particular number of the microneedles 70 within a microneedle array share a chamber.

As a non-limiting aspect of the invention, predetermined columns of microneedles 70 within a microneedle array can deliver an ECM composition from a separate microneedle base chamber 73. This allows for different ECM compositions to be used simultaneously.

### EXAMPLES

The following examples are provided to enable those skilled in the art to more clearly understand and practice the present invention. They should not be considered as limiting the scope of the invention, but merely as being illustrated as representative thereof.

### Example 1

Several canines were presented with trauma, wherein all layers of the skin and hair were removed, and the underlying tissue exposed primarily due to continuous traumatic contact with pavement. The wounds were cleaned and foreign objects removed.

The wounds also underwent debridement to remove all dead tissue and fibrosis that may have formed post-trauma. Xenografts formed from ECM (i.e. SIS) compositions were applied to the wound site of each animal, wherein the wound was fully covered and tacked down via single suture at various points across the area of the wound.

As the regeneration process started and the ECM material eventually absorbed into the new tissue, the wound started to form new tissue from the outside perimeter inwards. The natural regeneration process involved the integration and differentiation of healthy living cells along the perimeter of the wound into the ECM material. The wound subsequently reduced in size with the perimeter being regenerated and an area of the wound remaining in the center of the trauma. At this point, the wound was again debrided to remove any fibrosis or dead tissue and another ECM xenograft was applied to the wound using the same steps described above.

The debridement process and application of additional ECM xenografts was continued until the wound was fully replaced with native tissue.

It was observed that as new dermis appeared, so too did the original hair. It was also observed that the regenerated hair contained the same color and patterns as the pre-trauma hair, and also grew back to the same natural length and thickness.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A composition for inducing or promoting hair growth, comprising:
an extracellular matrix (ECM) composition, said ECM composition including an ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, ornamentum extracellular matrix, heart extracellular matrix and lung extracellular matrix.

2. The composition of claim 1, wherein said ECM material comprises a decellularized ECM material.

3. The composition of claim 1, wherein said ECM material includes at least one supplemental biologically active agent.

4. The composition of claim 3, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor-α (TNA-α), and placental growth factor (PLGF).

5. The composition of claim 3, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

6. The composition of claim 3, wherein said biologically active agent comprises an active agent selected from the group consisting of a collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

7. The composition of claim 3, wherein said biologically active agent comprises a pharmacological agent.

8. The composition of claim 7, wherein said pharmacological agent is selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

9. The composition of claim 7, wherein said pharmacological agent comprises a HMG-CoA reductase inhibitor.

10. The composition of claim 9, wherein said HMG-CoA reductase inhibitor is selected from the group consisting of atorvastatin, cerivastatin, fluvastatin, lovastatin, mevastatin, pitavastatin, pravastatin, rosuvastatin and simvastatin.

11. The composition of claim 1, wherein, when said composition is administered to a scalp of a subject, said composition further induces hair follicle differentiation and differentiation.

12. The composition of claim 1, wherein, when said composition is administered to a scalp of a subject, said composition further abates hair loss.

13. A method of inducing or promoting hair growth on the scalp of a subject, comprising the steps of:
providing an ECM composition including at least one ECM material selected from the group consisting of small intestine submucosa (SIS), urinary bladder submucosa (UBS), urinary basement membrane (UBM), liver basement membrane (LBM), stomach submucosa (SS), mesothelial tissue, subcutaneous extracellular matrix, large intestine extracellular matrix, placental extracellular matrix, omamentum extracellular matrix, heart extracellular matrix and lung extracellular matrix;
administering inciting event means to a target location on the scalp of the subject to induce at least one inciting event at said target scalp location; and
administering a therapeutically effective amount of said ECM composition to said target scalp location.

14. The method of claim 13, wherein said ECM material comprises a decellularized ECM material.

15. The method of claim 13, wherein said ECM material includes at least one additional biologically active agent.

16. The method of claim 15, wherein said biologically active agent comprises a growth factor selected from the group consisting of a platelet derived growth factor (PDGF), epidermal growth factor (EGF), transforming growth factor-α (TGF-α), transforming growth factor-β (TGF-β), fibroblast growth factor-2 (FGF-2), basic fibroblast growth factor (bFGF), vascular epithelial growth factor (VEGF), hepatocyte growth factor (1-IGF), insulin-like growth factor (IGF), nerve growth factor (NGF), platelet derived growth factor (PDGF), tumor necrosis factor-α (TNA-α), and placental growth factor (PLGF).

17. The method of claim 15, wherein said biologically active agent comprises a cell selected from the group consisting of a human embryonic stem cell, fetal cardiomyocyte, myofibroblast, mesenchymal stem cell, autotransplanted expanded cardiomyocytes, adipocyte, totipotent cell, pluripotent cell, blood stem cell, myoblast, adult stem cell, bone marrow cell, mesenchymal cell, embryonic stem cell, parenchymal cell, epithelial cell, endothelial cell, mesothelial cell, fibroblast, osteoblast, chondrocyte, exogenous cell, endogenous cell, hematopoietic stem cell, bone-marrow derived progenitor cell, myocardial cell, skeletal cell, fetal cell, undifferentiated cell, multi-potent progenitor cell, unipotent progenitor cell, monocyte, cardiac myoblast, skeletal myoblast, macrophage, capillary endothelial cell, xenogenic cell, allogenic cell and post-natal stem cell.

18. The method of claim 15, wherein said biologically active agent comprises an active agent selected from the group consisting of a collagen (types I-V), proteoglycans, glycosaminoglycans (GAGS), glycoproteins, cytokines, cell-surface associated proteins, cell adhesion molecules (CAM), endothelial ligands, matrikines, cadherins, immuoglobins, fibril collagens, non-fibrallar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, finulin II, integrins, transmembrane molecules, thrombospondins, ostepontins, and angiotensin converting enzymes (ACE).

19. The method of claim 15, wherein said biologically active agent comprises a pharmacological agent selected from the group consisting of antibiotics, antifungal agents, anti-viral agents, anti-pain agents, anesthetics, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants, antithrombic agents, DNA, RNA, modified DNA and RNA, NSAIDs, inhibitors of DNA, polypeptides, oligonucleotides, polynucleotides, nucleoproteins, and vasodilating agents.

20. The method of claim 19, wherein said pharmacological agent comprises a HMG-CoA reductase inhibitor selected from the group consisting of atorvastatin, cerivastatin, fluvastatin, lovastatin, mevastatin, pitavastatin, pravastatin, rosuvastatin and simvastatin.

21. The method of claim 13, wherein said inciting even means comprises piercing the scalp to a first depth.

22. The method of claim 13, wherein said inciting event means comprises the administration of energy to the scalp of the subject, said inciting event energy being selected from the group consisting of ultrasonic energy, radio frequency energy, laser energy, ultraviolet energy and infrared energy.
